(19) United States
(12) Patent Application Publication
(10) Pub. No.: US 2016/0270404 A1
(43) Pub. Date: Sep. 22, 2016

(54) METHODS AND COMPOSITIONS TO CONTROL UNDESIRABLE MICROORGANISMS IN FERMENTATION PROCESSES

(71) Applicants: Todd Wichmann, Loveland, OH (US); Flavea Trindade Reis, Sao Paulo (BR); Leane Oliveira, Sao Paulo (BR); Marcio Liboni, Sao Paulo (BR)

(72) Inventors: Todd Wichmann, Loveland, OH (US); Flavea Trindade Reis, Sao Paulo (BR); Leane Oliveira, Sao Paulo (BR); Marcio Liboni, Sao Paulo (BR)

(21) Appl. No.: 15/070,326

(22) Filed: Mar. 15, 2016

Related U.S. Application Data
(60) Provisional application No. 62/133,701, filed on Mar. 16, 2015.

(51) Int. Cl. A01N 59/02; A01N 37/02; C12P 7/06

(52) U.S. Cl. CPC A01N 59/02; C12P 7/06; A01N 37/02

(57) ABSTRACT

A method of controlling bacterial contamination by inhibiting bacterial growth in an aqueous fermentation solution employed in a sugar fermentation process is provided. The method includes the steps of: (a) introducing a surfactant composition into the aqueous fermentation solution; (b) introducing an acid into the aqueous fermentation solution in a sufficient quantity to provide a pH value in a range of about 4.5 or less; and (c) mixing the aqueous fermentation solution with yeast, wherein a combination of the surfactant composition and the pH provide antibacterial effects. The synergistic combination of the surfactant composition and the acidic pH of the aqueous fermentation solution are effective to control the levels of one or more of the undesirable bacteria and/or bacterial by-products, while the yeast (e.g., Saccharomyces cerevisiae) that convert sugar to ethanol remain suitably viable.

### CROSS REFERENCE TO RELATED APPLICATIONS

This application claims the benefit of U.S. Provisional Application Ser. No. 62/133,701, filed on Mar. 16, 2015, the disclosures of which are expressly incorporated by reference herein in their entirety.

### TECHNICAL FIELD

The present invention is generally directed to microbial control in fermentation processes. In particular, the present invention is directed to a method of reducing or controlling the concentration of undesirable microorganisms by inhibiting their growth.

### BACKGROUND

In the last decade, the use of ethanol as a transportation fuel has increased significantly. Ethanol production in the United States rose from approximately 6.4 billion liters in the year 2000 to over 37 billion liters in 2009. The number of ethanol plants increased from 54 in 2000 to 170 in 2009. Similar increases in production and plant construction have occurred in Latin America and Europe. In 2007, the United States Congress enacted the Energy Independence and Security Act (H.R. 6), which set the renewable fuel standard at 136 billion liters of ethanol by the year 2022. If this standard is to be met, the ethanol industry will continue to grow.

Currently both industrial ethanol (e.g., fuel) and beverage ethanol are produced on large scale from agricultural feedstocks by fermentation processes in which sugar is converted to ethanol and carbon dioxide by inoculant yeast. Many feedstocks can be used to provide the sugar for fermenting, including potentially, any starch or cellulosic material, which includes nearly all plants, as any starch or cellulose can be a precursor to sugar. Some of the common feedstocks particularly suitable for producing fuel ethanol include corn, milo, sorghum, sugar cane, sugar beets and molasses.

The feedstocks used for ethanol production are natural products. Therefore, a wide variety of microorganisms such as bacteria, fungi, and yeasts are likely to be naturally present in the feedstocks. Commercial fermentation process conditions are not completely sterile; hence these “contaminant microorganisms” will be present in the process. In commercial ethanol production, microorganisms of greatest concern are lactic acid-producing bacteria and acetic acid-producing bacteria. Such bacteria enter the process from several sources including raw materials, equipment, process water, air, and inoculant yeast, among others. Concentrations of such bacteria may increase in the process environment either through introduction with incoming materials (raw materials, water, air, yeast) or naturally proliferate as a result of conditions favorable to bacterial growth. The optimum atmosphere for yeast production is also extremely conducive to the growth of these bacteria. Organic acids produced by the bacteria inhibit the growth of yeasts and thus reduce ethanol production rate. The bacteria may also consume sugars and other nutrients intended for use by the yeast to produce desired products, rendering the entire process less economical.

The need for antibacterial treatments is increasing, not only because of the growth in production volume of ethanol but also the expansion in size of ethanol production facilities. Whereas a plant producing 150-200 million liters per year (MMIy) was considered a large facility just a few years ago, 380 MMIy (or more) facilities are today's industry standard. In fed-batch processes, the volume of individual fermentation batches has increased significantly. To accommodate this added capacity, the flow rate of feedstock (commonly known as “mash” once it has been prepared for entry into fermentation) into a fermentation system has increased from approximately 2000-3000 liters per minute to 4500-6000 liters per minute in the largest ethanol production facilities.

Many fermentation processes use antibiotics, such as minocin, as antibacterial compositions. Such use has become disfavored due to suspected development of antibiotic-resistant bacteria and accumulation of antibiotic residues in fermentation by-products. Antibiotic-resistant bacteria are a significant concern in human health.

Moreover, by-products of ethanol production include solids that are collected after distillation of the ethanol product. Such solids include distillers dried grains with solubles (DDGS) and distiller's wet grains with solubles (DWGS), both of which are subsequently sold as animal feed products. Accordingly, antibiotic residues can be present in these animal feed products, and thus, many countries are considering regulatory actions that would limit or eliminate the use of antibiotics for ethanol production.

Other fermentation processes have used non-antibiotic methods to control bacteria growth. For example, stabilized chlorine dioxide (SCD) has been utilized to prevent bacterial contamination in ethanol production. SCD is added prior to the onset of significant bacterial growth in ethanol production, as a preventive rather than as a remedial measure. The growth of contaminant bacteria prior to and during the fermentation of sugar to alcohol is thus substantially prevented, creating conditions that enhance growth of inoculant yeast and enable inoculant yeast to produce ethanol without inhibition by organic acids produced by the bacteria.

Urea hydrogen peroxide (UHP) has also been utilized to prevent bacterial growth in fermentation processes. UHP is available commercially in only limited quantities as this adduct has production and storage issues. UHP is added to the process prior to the introduction of yeast, thus eliminating a substantial population of bacterial contaminants, and allowing inoculant yeast to convert fermentation feedstocks into ethanol unhindered. UHP can only be utilized prior to the introduction of inoculant yeast, as the yeast is capable of metabolizing, and thus neutralizing the UHP and rendering it inactive against bacteria. UHP is also not stable during storage conditions commonly encountered in the ethanol industry.

Although some methods are known, there remains demand for improved methods, for addressing contaminant microorganisms in the fermentation industry, both for carbohydrate-containing feedstocks and in fermentation processes. An improved method should preferably be antibiotic-free and not result in residues that accumulate in fermentation by-products or give rise to antibiotic-resistant bacteria. The method should be efficacious at conditions encountered in the fermentation industry.

Accordingly, there remains a need for new methods for controlling undesirable microorganism concentration in fermentation processes.

### SUMMARY

In one embodiment of the present invention, a method of controlling microbial contamination by inhibiting the growth of bacteria in an aqueous fermentation solution employed in a sugar fermentation process is provided. The method comprising the steps of: (a) introducing an effective amount of a surfactant composition into the aqueous fermentation solution; (b) introducing an acid into the aqueous fermentation solution in a sufficient quantity to provide a pH value in a range of about 4.5 or less; and (c) mixing the aqueous fermentation solution with yeast, wherein a combination of the surfactant composition and the pH provide antibacterial effects. The surfactant composition and/or the acid may be added to the fermentable sugar or process water prior to introducing each of these to the fermentation vessel. Alternatively the surfactant composition, acid, and/or yeast may be added directly to the fermentation vessel. The surfactant composition may be formulated with nutrients, minerals, and other ingredients, such as acids, that might prove beneficial to the overall operation of a fermentation process. Accordingly, in yet another embodiment, the surfactant composition and the acid components may combined to form a single composition, which may then be combined with the fermentable sugar or process water, or added directly to the fermentation vessel.

In accordance with another embodiment of the present invention, an aqueous fermentation solution comprising water, sugar, yeast, acid, and an antibacterial composition comprising a surfactant composition, wherein the surfactant composition is present in a sufficient quantity to provide a concentration of a surfactant in the aqueous fermentation solution equal to about 5 ppm or more; and wherein the aqueous fermentation solution has a pH value in a range of about 4.5 to about 1.0.

### DETAILED DESCRIPTION

In accordance with embodiments of the present invention, it has been discovered that the undesirable bacteria levels in a sugar fermentation process can be improved by incorporating a surfactant composition in an aqueous fermentation solution at a pH equal to about 4.5 or less. Exemplary undesirable bacteria that may interfere during sugar fermentation include Gram (+) and Gram (−) bacteria such as Lactobacillus spp., Leuconostoc spp., Pediococcus spp., Staphylococcus spp., Bacillus spp., and Clostridium spp. The combination of the surfactant composition and the acidic aqueous fermentation solution are effective to control the levels of one or more of the undesirable bacteria and/or bacterial by-products, while the yeast (e.g., Saccharomyces cerevisiae) that convert sugar to ethanol remain suitably viable. Without being bound to a particular theory, the surfactant composition has an antibacterial effect at an acidic pH by increasing the permeability of the bacterial cell wall, leading to a reduction of intracellular pH, thereby changing the electrolyte balance and killing the bacteria. The yeast cell wall, which includes chitin, is different from that of bacteria and is not compromised by the surfactant composition when used as described herein.

The surfactant can be a single type, but may also be a combination of two or more surfactants. It is common to characterize surfactants by a hydrophile-lipophile balance value, also known as HLB value. Surfactants with a low HLB are more lipid loving and thus tend to make a water in oil emulsion while those with a high HLB are more hydrophilic and tend to make an oil in water emulsion. When combinations of surfactants are used, the weighted average of the individual surfactant components is used to calculate the HLB of the combination. An exemplary range of HLB values for individual surfactants or combination of surfactants combination is about 6 to about 50.

In one embodiment, the surfactant composition includes an anionic surfactant, a cationic surfactant, a zwitterionic surfactant, a nonionic surfactant, or combinations thereof. In another embodiment, the surfactant composition includes an anionic surfactant, a nonionic surfactant, or combinations thereof. Exemplary surfactants that are useful in the present invention include, without limitation, ethoxylated alcohols, ethoxylated carbohydrates, ethoxylated vegetable oils, polyethyleneglycols (PEG), polypropylene glycols (PPG), monoesters and diesters of PEG and PPG, alkyl (linear or branched) sulfates, ethoxylated amines, fatty acids, ethoxylated fatty acids, fatty amides, fatty diethanolam ides, saponins, sugar-based surfactants, and the like.

Examples of specific surfactants, and commercial sources, include oleyl alcohol 10 EO (Ethox Chemical), polysorbate 20 (Tween 20—Uniqema), stearyl alcohol 20 EO (Ethox Chemical), castor oil 80 EO (Ethox Chemical), castor oil 30 EO (Ethox Chemical), polyethylene glycol (PEG) 400 Dioleate (Ethox Chemical), tallow amine 5 EO (Akzo Nobel), Burco TME-S (Burlington Chemical), coconut diethanolamide (Ethox Chemical), Ethfac 161 (Ethox Chemical), cocoamine 2 EO (Akzo Nobel), cocoamine 5 EO (Akzo Nobel), Dowanol DB (Dow Chemical), Demulse DLN 532 CE (Deforest Enterprises), Tween 80 (Uniqema), Demulse DLN 622 EG (Deforest Enterprises), Span 20 (Uniqema), Diacid 1550 (Westvaco), decyl alcohol 4 EO (Ethox Chemical), dipropyleneglycol methyl ester (Dow Chemical), linear or branched alkyl sulfates such as sodium lauryl sulfate SLS, sodium dodecyl sulfate SDS, or sodium xylenesulfonate SXS (Dow Chemical), and Tergitol NP6 (Dow Chemical).

In accordance with other embodiments of the present invention, the surfactant composition may further include other additional ingredients, such as acid or base stable anionic surfactants, sequestrants, builders, buffers, preservatives, salts, sulfates, fatty acids, unsulfonated alcohols, anticaking materials such as silicon dioxide, and/or fluid carriers.

Additional Surfactants: Optionally, acid or base stable anionic surfactants can be employed, as allowed by the United States Code of Federal Regulations (CFR), Title 21, Section 173.315. Described in the CFR are phosphate esters of ethylene and/or ethylene/propylene oxide adducts of aliphatic alcohols, dioctyl sulfosuccinate, or 2-ethylhexyl sulfate, typically but these materials suffer from lack of stability at either acid or basic conditions.

Sequestrant/builder: An organic polycarboxylic acid, or salt thereof, e.g., citric acid, may be used as a sequestrant/builder in an acidic formulation or sodium and/or potassium citrate may be used in a basic formulation. Citric acid, sodium citrate, and potassium citrate are standard items of commerce. Other organic poly carboxylic acids, especially those that are GRAS, such as tartaric, malic, etc. acids, can also be used. When formulating the basic formulations herein, it is preferred to use the potassium salt, as compared with the sodium salt, to provide ease of formulatability. Complex phosphates can also be used, but are generally avoided due to regulatory considerations.

Buffer: Toxicologically-acceptable acidic or basic buffers can be used in the compositions herein to maintain product pH in the acid or base range. For ease of during formulation, it is preferred that such acidic and basic buffers be in their potassium salt form. Citric acid is a preferred acid pH buffer, and in the basic pH systems, potassium citrate is a preferred dispersant. Potassium carbonate is a convenient and preferred basic pH buffer. Sodium bicarbonate is a highly desirable material to add to the compositions of this invention as a part of the buffering system since it is readily available as baking soda in food grade and is therefore relatively inexpensive, while providing a highly desirable purity to the composition. Surfactant compositions formulated with a mixture of potassium and sodium cations in molar ratios of from about 1:1 to about 10:1, preferably from about 2:1 to about 8:1, more preferably from about 4:1 to about 5:1 potassium to sodium, e.g., as provided by mixtures of potassium hydroxide (hydrate) and sodium bicarbonate, have desirable rheological properties.

The ability of the surfactant compositions containing mixtures of both sodium and potassium cations to shear thin can be important to promote easy dispensing, especially when the compositions are formulated as liquids.

pH: The pH of the surfactant compositions is not particularly limited except with respect to maintaining chemical and physical stability of the surfactant composition itself. However, pH values not greater than about 12 or pH values not less than about 1.0 are generally preferred.

Preservative: The strategy of formulating the present compositions at either high or low pH is based on the reduced tendency for biological growth of contaminants, such as bacteria, fungi, or molds, at either high pH (>9) or low pH (<5). At neutral pH, an increased reliance on preservatives is required to insure the lack of biological growth through contamination in making or in use. The acidic surfactant compositions herein may contain a minimal amount, typically from about 0.01% to about 0.2% by weight, of a toxicologically-acceptable preservative in order to prevent the growth of fungi, bacteria or like in the product on storage. Standard food-grade preservatives such as potassium sorbate/sorbic acid and/or sodium benzoate/benzoic acid, or mixtures thereof, are also suitable for such purposes. For example, from about 0.01% to about 0.2% of benzoic acid or its sodium or potassium salts can be used. In general, the basic pH compositions herein do not require a preservative, although one can be added if desired.

Fluid Carrier: In accordance with an embodiment, the surfactant composition may be formulated as a liquid formulation. In liquid formulations, a major proportion (e.g., about 50% to about 98%, by weight) of the composition may comprise water as a solubilizing carrier for the ingredients. Water and ethanol combinations can also be employed. Other compatible, water-soluble, low molecular weight solvents can also be used, so long as the solvents do not detrimentally affect the fermentation process.

With respect to the sugar-containing fermentation feedstock that makes up the aqueous fermentation solution, many different feedstocks can be used to provide the sugar for fermenting, including potentially, any starch or cellulosic material. Some of the common feedstocks particularly suitable for producing fuel ethanol include corn, milo, sorghum, sugar cane, sugar beets, and molasses. Some of the common feedstocks for potable ethanol solutions include, but are not limited to, fruit such as berries, grapes, pears, peaches, or apples; corn, grains; potatoes; agave; or the like. In an embodiment, the sugar-containing fermentation feedstock is derived from sugar cane. In another embodiment, the sugar-containing fermentation feedstock is derived from corn. In yet another embodiment, the sugar-containing fermentation feedstock is derived from fruit, corn, grains, potatoes, agave, or combinations thereof.

In an embodiment of the method of the invention, surfactant combination is introduced into the aqueous fermentation solution in a fermentation process for the production of ethanol. For convenience, the surfactant composition may be added as a liquid, with batch, semi-batch, or continuous addition. Accordingly, a solid or powder formulation may be combined with a suitable liquid dispersing medium, e.g., water, prior to combining with the aqueous fermentation solution.

In accordance with embodiments of the present invention, the sugar fermentation is carried out as an acidic aqueous sugar fermentation. In an embodiment, the acidic aqueous sugar fermentation is carried out with the pH of the aqueous fermentation solution at a value of about 4.5 or less. For example, the pH of the aqueous fermentation solution may be about 4.0 or less, or about 3.5 or less, or about 3.0 or less, about 2.5 or less, or from about 1.0 to about 4.5 or from about 1.0 to about 3.5, or from about 1.0 to about 2.5. Accordingly, in embodiments where the surfactant composition does not contain a sufficient quantity of acid to provide the desired pH for the aqueous fermentation solution, acid may be added in a sufficient quantity to achieve the desired pH value. Non-limiting acids suitable for acidifying the aqueous fermentation solution include sulfuric acid, hydrogen sulphate, levulinic acid, caprylic acid, caproic acid, citric acid, eugenol, adipic acid, tartaric acid, fumaric acid, lactic acid, phosphoric acid, hydrochloric acid, succinic acid, malic acid and sorbic acid, acetic acid, and combinations thereof. In an embodiment, the acid includes sulfuric acid. In another embodiment, the acid includes citric acid.

In an embodiment, the combination of the surfactant composition and the acid is added to the aqueous fermentation solution before inoculation with the yeast. The combination of the surfactant composition and the acid can also be added to the fermentation medium during the first one-third of the fermentation cycle. As used herein, the term “first one-third of the fermentation cycle” refers to the first ⅓ of the total time of fermentation. In another embodiment, the combination of the surfactant composition and the acid is added to the fermentation medium during the middle one-third of the fermentation cycle, or the combination of the surfactant composition and the acid is added to the fermentation medium during the final one-third of the fermentation cycle. As used herein, the term “middle one-third of the fermentation cycle” refers to the middle ⅓ of the total time of fermentation, and the term “final one-third of the fermentation cycle” refers to the last ⅓ of the total time of fermentation. In some embodiments, the combination of the surfactant composition and the acid may be added to the fermentation medium in two or more increments during the fermentation cycle. The duration of the fermentation cycle can vary depending on a variety of factors, such as the fermentation equipment, yeast strain, temperature, concentration of sugar, concentration of ethanol, etc. In one non-limiting embodiment, the fermentation cycle is 12 hours in a 600,000 liter to 1,000,000 liter fermentation vessel at 32° C.-36° C. at an initial yeast concentration of about 10% of the final volume of the fermentation tank, and wort composed of water and fermentable sugars such as the sugars in sugarcane juice and/or molasses.

Bacterial contamination appears to reduce the sugar fermentation process productivity, by reducing yeast growth, viability, and fermentation capacity. Thus, in accordance with embodiments of the present invention, the surfactant composition is present in the aqueous fermentation solution, having a pH value in a range of about 4.5 to about 1.0, in an amount that is effective to decrease the bacteria count and/or the yield of one or more benchmark bacteria products. Lactic acid-producing bacteria (LAB) are very abundant in the bioethanol process possibly because of their tolerance to ethanol, low pH and high temperature. Lactic and acetic acids produced by LAB may interfere in the yeast metabolism, which is detrimental to the sugar fermentation process. For example, lactic acid is known to detrimentally effect yeast conversion of sugar to ethanol.

Accordingly, in one aspect, embodiments of the present invention may reduce the bacterial count over a twelve (12) hour fermentation cycle by a factor of 10 or more, as compared to a single twelve (12) hour fermentation cycle of the aqueous fermentation solution at a pH equal to about 2 (acidified with sulfuric acid) and devoid of the surfactant composition. According to another aspect, the formation of lactic acid may be reduced by 50% or more, relative to a single twelve (12) hour fermentation cycle of the aqueous fermentation solution at a pH equal to about 2 (acidified with sulfuric acid) and devoid of the surfactant composition.

As noted above, the surfactant composition may be a solid or liquid formulation having a plurality of components. Accordingly, the surfactant(s) content of the surfactant composition may vary depending on the specific formulation. However, in accordance with embodiments of the present invention, the surfactant composition is added to the aqueous fermentation solution in an amount to provide a surfactant concentration that is effective, in combination with the pH of about 4.5 or less, to provide antibacterial effects. In an embodiment, the surfactant composition added to the aqueous fermentation solution in an amount to provide a surfactant concentration equal to about 5 ppm or more, by weight. In another embodiment, the surfactant composition is added to the aqueous fermentation solution in an amount to provide a surfactant concentration equal to about 10 ppm or more, by weight. For example, the surfactant concentration may be about 20 ppm or more, about 30 ppm or more, about 40 ppm or more, about 50 ppm or more, about 75 ppm or more, about 100 ppm, about 150 ppm, about 200 ppm, about 250 ppm, about 300 ppm, about 350 ppm, about 400 ppm, about 450 ppm, or about 500 ppm or more. In yet another example, the surfactant composition may added to the aqueous fermentation solution in an amount to provide a surfactant concentration equal to about 25 ppm to about 500 ppm by weight, or about 75 ppm to about 350 ppm by weight, or about 50 ppm to about 150 ppm by weight.

The term “effective amount” and “antibacterially effective amount”, with respect to the surfactant composition, includes an amount capable of performing the function or having the property or result desired. Effective amount or antibacterially effective amount, for example, would be an amount which results in a significant decrease in the concentration of bacteria as compared to where no surfactant composition is employed. An effective amount or antibacterially effective amount also will be relatively non-toxic to yeast but provide antibacterial benefits. One embodiment of an effective amount or antibacterially effective amount is an amount sufficient to suppress bacterial contamination while maintaining an acceptable level of yeast viability, such as at least 50% yeast viability. In an embodiment, an effective amount or antibacterially effective amount provides a concentration of a surfactant, or surfactants, (“surfactant”) from the surfactant composition in the aqueous fermentation solution equal to about 5 ppm or more. Accordingly, an effective amount of the surfactant composition at a specific pH value in the range of about 4.5 to about 1.0 may be determined by one having ordinary skill in the art by routine experimentation.

In another embodiment, the surfactant composition is present in an effective amount in the acidic fermentation solution having a pH value in a range of about 4.5 to about 1.0 to provide antibacterial effects, wherein the acidic aqueous fermentation solution is substantially free of any added antibiotic agents, any added guanidine compounds, and/or any added oxidants selected from chlorine dioxide, stabilized chlorine dioxide, or peroxide compounds. In another embodiment, the acidic aqueous fermentation solution is substantially free of any terpene or terpenoid compounds.

The following embodiments are also contemplated:

1. A method of controlling bacterial contamination by inhibiting bacterial growth in an aqueous fermentation solution employed in a sugar fermentation process, the method comprising the steps of:

(a) introducing an effective amount of a surfactant composition into the aqueous fermentation solution;

(b) introducing an acid into the aqueous fermentation solution in a sufficient quantity to provide a pH value in a range of about 4.5 or less; and

(c) mixing the aqueous fermentation solution with yeast, wherein a combination of the surfactant composition and the pH provide antibacterial effects.

2. The method of clause 1, wherein said steps are performed sequentially.

3. The method of clause 1 or 2, wherein the acid is selected from the group consisting of sulfuric acid, hydrogen sulphate, levulinic acid, caprylic acid, caproic acid, citric acid, adipic acid, tartaric acid, fumaric acid, lactic acid, phosphoric acid, hydrochloric acid, succinic acid, malic acid, sorbic acid, acetic acid, and combinations thereof.

4. The method of one of clauses 1 to 3, wherein the pH value is in a range of about 4.5 to about 1.0.

5. The method of one of clauses 1 to 4, wherein the pH value is in a range of about 3.5 to about 1.0.

6. The method of one of clauses 1 to 5, wherein the surfactant composition is added in a sufficient quantity to provide a concentration of a surfactant equal to about 5 ppm or more.

7. The method of one of clauses 1 to 6, wherein the surfactant composition is added in a sufficient quantity to provide a concentration of a surfactant equal to about 25 ppm or more.

8. The method of one of clauses 1 to 7, wherein the surfactant composition is added in a sufficient quantity to provide a concentration of a surfactant equal to about 50 ppm or more.

9. The method of one of clauses 1 to 8, wherein the surfactant composition is added in a sufficient quantity to provide a concentration of a surfactant equal to about 75 ppm or more.

10. The method of one of clauses 1 to 9, wherein the surfactant composition is added in a sufficient quantity to provide a concentration of a surfactant in a range from about 75 ppm to about 350 ppm.

11. The method of one of clauses 1 to 10, wherein the surfactant composition comprises a non-ionic surfactant, an anionic surfactant, or a combination thereof.

12. The method of one of clauses 1 to 11, wherein the surfactant composition comprises a surfactant selected from a group consisting of ethoxylated alcohols, ethoxylated carbohydrates, ethoxylated vegetable oils, polyethyleneglycols (PEG), polypropylene glycols (PPG), monoesters and diesters of PEG and PPG, linear or branched alkyl sulfates, ethoxylated amines, organosulfates, fatty acids, ethoxylated fatty acids, fatty amides, fatty diethanolamides, saponin, sugar-based surfactants, and combinations thereof.

13. The method of clause 11, wherein the surfactant composition further comprises one or more acidic compounds.

14. The method of one of clauses 1 to 13, wherein an undesirable bacterial concentration level in the aqueous fermentation solution after a twelve hour fermentation cycle is reduced by at least a factor of 10, relative to a single twelve hour fermentation cycle of the aqueous fermentation solution at a pH equal to about 2 acidified with sulfuric acid and devoid of the surfactant composition.

15. The method of one of clauses 1 to 14, wherein an undesirable bacterial by-product concentration level in the aqueous fermentation solution after a twelve hour fermentation cycle is reduced by 50% or more, relative to a single twelve hour fermentation cycle of the aqueous fermentation solution at a pH equal to about 1.0 acidified with sulfuric acid and devoid of the surfactant composition.

16. An aqueous fermentation solution comprising water, sugar, yeast, acid, and an antibacterial composition comprising a surfactant composition, wherein the surfactant composition is present in a sufficient quantity to provide a concentration of a surfactant equal to about 5 ppm or more; and wherein the aqueous fermentation solution has a pH value in a range of about 4.5 to about 1.0.

17. A method of controlling bacterial contamination by inhibiting bacterial growth in an acidic aqueous sugar fermentation comprising mixing an aqueous fermentation solution with yeast, said aqueous fermentation solution comprising an effective amount of a surfactant composition.

18. A method of controlling bacterial contamination by inhibiting bacterial growth in an acidic aqueous sugar fermentation comprising mixing an aqueous fermentation solution with an effective amount of a surfactant composition, said aqueous fermentation solution comprising yeast.

19. A method of controlling bacterial contamination by inhibiting bacterial growth in an acidic aqueous sugar fermentation comprising mixing an aqueous fermentation solution with a mixture comprising an effective amount of a surfactant composition and yeast.

20. A method of controlling bacterial contamination by inhibiting bacterial growth in an acidic aqueous sugar fermentation comprising mixing an aqueous fermentation solution, an effective amount of a surfactant composition, and yeast.

21. A method of controlling bacterial contamination by inhibiting bacterial growth in an aqueous sugar fermentation comprising adjusting the pH of an aqueous fermentation solution to about 4.5 or less, said aqueous fermentation solution comprising an effective amount of a surfactant composition and yeast.

22. A method of controlling bacterial contamination by inhibiting bacterial growth by inhibiting bacterial growth in an aqueous sugar fermentation comprising fermenting a mixture comprising an aqueous fermentation solution, yeast, and an effective amount of a surfactant composition, at a pH of about 4.5 or less.

23. An acidic aqueous fermentation solution comprising water, sugar, yeast, and a surfactant composition, wherein the surfactant composition is present in an antibacterially effective amount.

24. An acidic aqueous fermentation solution comprising water, sugar, yeast, and a surfactant composition, wherein the surfactant composition is present in a quantity sufficient to provide a concentration of surfactant of about 5 ppm or more.

25. The method of one of clauses 17 to 22, wherein the pH of the aqueous fermentation solution is adjusted to a desired pH with an acid selected from the group consisting of sulfuric acid, hydrogen sulphate, levulinic acid, caprylic acid, caproic acid, citric acid, adipic acid, tartaric acid, fumaric acid, lactic acid, phosphoric acid, hydrochloric acid, succinic acid, malic acid, sorbic acid, acetic acid, and combinations thereof.

26. The method of one of clauses 17 to 22, wherein the aqueous fermentation solution has a pH value in a range of about 4.5 to about 1.0.

27. The method of one of clauses 17 to 22, wherein the aqueous fermentation solution has a pH value in a range of about 3.5 to about 1.0.

28. The method of one of clauses 17, 18, 19, 20, 21, 22, 25, 26, or 27, wherein the surfactant composition is added in a sufficient quantity to provide a concentration of a surfactant equal to about 5 ppm or more.

29. The method of one of clauses 17, 18, 19, 20, 21, 22, 25, 26, or 27, wherein the surfactant composition is added in a sufficient quantity to provide a concentration of a surfactant equal to about 25 ppm or more.

30. The method of one of clauses 17, 18, 19, 20, 21, 22, 25, 26, or 27, wherein the surfactant composition is added in a sufficient quantity to provide a concentration of a surfactant equal to about 50 ppm or more.

31. The method of one of clauses 17, 18, 19, 20, 21, 22, 25, 26, or 27, wherein the surfactant composition is added in a sufficient quantity to provide a concentration of a surfactant equal to about 75 ppm or more.

32. The method of one of clauses 17, 18, 19, 20, 21, 22, 25, 26, or 27, wherein the surfactant composition is added in a sufficient quantity to provide a concentration of a surfactant in a range from about 75 ppm to about 350 ppm.

33. The method of one of clauses 17, 18, 19, 20, 21, 22, 25, 26, 27, 28, 29, 30, 31, or 32, wherein the surfactant composition comprises a non-ionic surfactant, an anionic surfactant, or a combination thereof.

34. The method of one of clauses 17, 18, 19, 20, 21, 22, 25, 26, 27, 28, 29, 30, 31, 32, or 33, wherein the surfactant composition comprises a surfactant selected from a group consisting of ethoxylated alcohols, ethoxylated carbohydrates, ethoxylated vegetable oils, polyethyleneglycols (PEG), polypropylene glycols (PPG), monoesters and diesters of PEG and PPG, linear or branched alkyl sulfates, ethoxylated amines, organosulfates, fatty acids, ethoxylated fatty acids, fatty amides, fatty diethanolam ides, saponin, sugar-based surfactants, and combinations thereof.

35. The method of one of clauses 17, 18, 19, 20, 21, 22, 25, 26, 27, 28, 29, 30, 31, 32, 33, or 34, wherein an undesirable bacterial concentration level in the aqueous fermentation solution after a twelve hour fermentation cycle is reduced by at least a factor of 10, relative to a single twelve hour fermentation cycle of the aqueous fermentation solution at a pH equal to about 2 acidified with sulfuric acid and devoid of the surfactant composition.

36. The method of one of clauses 17, 18, 19, 20, 21, 22, 25, 26, 27, 28, 29, 30, 31, 32, 33, 34, or 35, wherein an undesirable bacterial by-product concentration level in the aqueous fermentation solution after a twelve hour fermentation cycle is reduced by 50% or more, relative to a single twelve hour fermentation cycle of the aqueous fermentation solution at a pH equal to about 1.0 acidified with sulfuric acid and devoid of the surfactant composition.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims, which follow the examples.

### Example 1

### Experimental

Method for determining bacterial count: The methodology used the dyes methylene blue and nilo, which distinguish live cells from dead cells. The dead cells are stained a blue color while the live cell remain colorless. The result in cells/mL is obtained by: Counted cells/counted fields*microscope factor*1/volume of the sample*dilution factor.

Method for determining lactic acid (and units): The concentrations of lactate were determined by a chromatographic method, using an ionic exchange column with a KOH solution as the eluent and a conductivity detector. Values obtained are in mg/L.

Method for calculating yeast viability: Yeast viability can be determined by two methods. The first method is conventional microscopy, where cells are colored with the dye erythrosin and counted. The dead cells are stained a pink color while the live cells remain colorless. The result in % is obtained by dividing the number of live cells by the sum of number of live cells and dead cells, multiplied by 100. The second method is plate counting. Aliquots of the fermented medium, taken simultaneous to the samples used for counting method, are serially diluted, and an appropriate volume was seeded on YEPD medium containing 1.5% agar and incubated at 30° C. for 3 days or until the appearance of colonies that could be visually counted. YEPD medium contained yeast extract (1%), peptone (1%) and dextrose (2%) at a pH of approximately 6.0. The results were expressed in CFU/mL (colony forming units per mL of fermentation broth).

Tables 1 to 5 contain results from a development study conducted to evaluate impact of pH values, antimicrobial concentration, and acid source on the ethanol fermentation process in laboratory scale. The same molasses source (for the fermentation process) and Lactobacillus strains (for inoculation) were used. The test article was evaluated considering different combinations of 3 doses, 3 pH values and 2 acids sources in order to understand the impact of these parameters on the ethanol fermentation process. This was a blinded product development study. A repeated measure design was used to evaluate one test article with two center point replications per acid source per week, two negative control replications per week, and different combinations of extreme doses and pH with both sulfuric and citric acids. Five (5) fermentation cycles were utilized to verify consistency of fermentation parameters, including antimicrobial capacity of treatments. This experiment was carried out over 5 fermentation cycles, simulating conditions imposed by industrial fermentation. The first fermentation cycle started with addition of approximately 10⁸ bacteria cells/mL, and no bacteria recontamination was allowed (sterilized substrate was utilized). To simulate the fermentation process, test article was added in a small volume mash where bacteria and yeast were highly concentrated (30-35% yeast concentration). Acid was added to reach the desired pH level. At this stage the total volume in the vat was 12 mL, half of which was composed of water and test article. The test article was added to the fermentation vat in a pre-established volume to achieve the final concentration in a 40 mL. In order to simulate the industrial ethanol fermentation process, the methodology used for this trial followed specific conditions: 1—Simulation of fed batch fermentation, observing feeding time and fermentation; 2—Simulation of a concentration in the centrifuge around 60-70% of yeast and bled about 1-5% of yeast (mixed with bacteria); 3—Separation of yeast at the end of fermentation with centrifugal force equal to that imposed in the industrial process (800×g); 4—Simulation of recycling fermentation conditions (usually five cycles allow fermentation show the effectiveness of an antimicrobial agent); 5—Use industrial substrate (based on molasses) with Total Reducing Sugars (TRS) content resulting around 8-9% ethanol at the end of fermentation; 6—Use of an industrial Saccharomyces cerevisiae strain that is normally found in the wort; 7—Contamination of fermentation with a minimum of 5 recently isolated Lactobacillus strains (homo and heterofermentative) that can simulate an industrial contamination challenge. The medium containing a sterile industrial substrate (molasses) and a Total Reducing Sugars (TRS) substrate was introduced to the fermentation vat resulting in medium with 8 to 9% ethanol at the end of fermentation. The fermentation was artificially contaminated with a minimum of 5 recently isolated Lactobacillus strains (homo and hetero fermentative), which simulated an industrial contamination challenge. Yeast Preparation: 1—Reactivation of industrial strain PE-2 (kept under ultra-freezer cooling at −80° C.), by culturing in 5 mL of YEPD medium for 48 hours at 30° C. 2—The medium with yeast was diluted in sugar cane molasses (sterile) containing 10% TRS (defined as molasses medium) and the growth was maintained for 48 hours at 30° C. 3—Molasses medium containing 10% TRS was added (doubling the volume of yeast suspension every 24-48 hours) to obtain the necessary biomass for the test (ca. 50 g of fresh yeast), which was collected by centrifugation at 800×g. Bacteria Preparation: 1—At least five strains of Lactobacillus (recently isolated from various distilleries and stored in −80° C.), comprising both types of metabolism (homo and heterofermentative), were added in 3 ml of MRS medium and cultivated at 32° C. for 48 hours. 2—The MRS medium with the Lactobacilli was added to 100 mL molasses medium with 3% of TRS and incubated at 32° C. for 48 hours to generate biomass acclimated to a molasses medium in sufficient quantity to allow a bacterial contamination of approximately 10⁸ cells/m L. Preparation of Yeast Contaminated with Bacteria: The yeast biomass was contaminated with bacteria (at the level of 10⁸ rod shaped cells per mL) and subjected to two fermentation cycles for a previous stabilization of the contamination (bacterial adaptation to the fermentation environment). Conduction of fermentations with cell recycle: The fermentation was conducted to simulate the conditions of fed batch industrial process with recycling of cells. The yeast suspension (30-35% yeast concentration) was prepared and treated with acid and test article for 1 hour. This treated yeast was gradually fed substrate (over a period of 4 hours) to obtain a final ethanol concentration between 8 and 9%. The fermentation rate was estimated by weight loss (evolution of carbon dioxide). At the end of fermentation the yeast was separated by centrifugation (800×g for 15 minutes), weighed and reused in a subsequent fermentation, comprising 5 cycles of fermentation.

TABLE 1
Data from First Fermentation Cycle Experiments.

| | | | | First Cycle | | | | |
|---|---|---|---|---|---|---|---|---|
| | | | | Bacteria | | Lactic | | |
| | Acid | | Dose | Count | | Acid | Yeast | |
| | Source | pH | (ppm) | (cells/mL) | L | (mg/L) | Viab (%) | Yeast CFU/mL |
| 1-1 | sulfuric | 3.3 | 325 | 1.86E+07 | 5.10E+07 | 1.939 | 70.3% | 2.23E+08 |
| 1-2 | citric | 2 | 500 | 4.09E+06 | 6.67E+06 | 1.379 | 50.2% | 2.00E+08 |
| 1-3 | sulfuric | 4.5 | 150 | 1.72E+08 | 6.10E+08 | 4.512 | 90.40% | 2.80E+08 |
| 1-4 | sulfuric | 2 | 0 | 2.22E+08 | 6.63E+08 | 5.626 | 96.0% | 2.83E+08 |
| 1-5 | citric | 3.3 | 325 | 4.64E+07 | 7.67E+07 | 2.185 | 72.0% | 1.70E+08 |
| 1-6 | citric | 4.5 | 150 | 2.76E+08 | 7.03E+08 | 4.534 | 94.4% | 3.00E+08 |
| 1-7 | sulfuric | 2 | 150 | 5.41E+07 | 1.40E+08 | 2.171 | 89.5% | 2.67E+08 |
| 1-8 | sulfuric | 2 | 500 | 1.27E+07 | 2.53E+07 | 1.640 | 53.7% | 2.20E+08 |
| 1-9 | citric | 3.3 | 325 | 1.09E+07 | 3.53E+07 | 1.962 | 68.4% | 2.33E+08 |
| 1-10 | sulfuric | 2 | 0 | 2.24E+08 | 8.07E+07 | 5.589 | 93.4% | 2.97E+08 |
| 1-11 | citric | 4.5 | 500 | 1.36E+07 | 2.67E+07 | 1.868 | 61.4% | 1.90E+08 |
| 1-12 | sulfuric | 4.5 | 500 | 1.13E+07 | 4.30E+07 | 2.180 | 57.1% | 1.97E+08 |
| 1-13 | sulfuric | 3.3 | 325 | 2.50E+07 | 6.60E+07 | 2.173 | 74.8% | 2.07E+08 |
| 1-14 | citric | 2 | 150 | 1.72E+07 | 2.67E+06 | 1.188 | 83.9% | 3.00E+08 |

Discussion of Table 1—After completion of the first cycle, it was observed a decrease in bacteria cell count (cells/mL and CFU/mL) and yeast cell viability (%) at the 325 and 500 ppm dose level. Other parameters were not influenced by treatment.

TABLE 2
Data from Second Fermentation Cycle Experiments.

| | | | | 2nd Cycle | | | |
|---|---|---|---|---|---|---|---|
| | | | | Bacteria | Lactic | Yeast | |
| | Acid | | Dose | Count | Acid | Viab | Yeast |
| | Source | pH | (ppm) | (cells/mL) | (mg/L) | (%) | CFU/mL |
| 1-1 | sulfuric | 3.3 | 325 | 1.18E+07 | 407 | 72.5% | 2.67E+08 |
| 1-2 | citric | 2 | 500 | 2.27E+06 | 315 | 51.8% | 1.83E+08 |
| 1-3 | sulfuric | 4.5 | 150 | 2.09E+07 | 2.565 | 90.7% | 2.40E+08 |
| 1-4 | sulfuric | 2 | 0 | 1.25E+08 | 3.268 | 97.7% | 2.97E+07 |
| 1-5 | citric | 3.3 | 325 | 5.00E+06 | 481 | 75.8% | 2.28E+08 |
| 1-6 | citric | 4.5 | 150 | 5.96E+07 | 2.711 | 89.6% | 3.17E+08 |
| 1-7 | sulfuric | 2 | 150 | 1.24E+07 | 494 | 85.7% | 2.93E+08 |
| 1-8 | sulfuric | 2 | 500 | 8.64E+06 | 334 | 56.0% | 1.90E+08 |
| 1-9 | citric | 3.3 | 325 | 6.37E+06 | 448 | 66.6% | 2.27E+08 |
| 1-10 | sulfuric | 2 | 0 | 2.77E+08 | 3.341 | 94.0% | 3.07E+08 |
| 1-11 | citric | 4.5 | 500 | 5.00E+06 | 416 | 49.2% | 1.93E+07 |
| 1-12 | sulfuric | 4.5 | 500 | 4.09E+06 | 449 | 48.7% | 1.93E+08 |
| 1-13 | sulfuric | 3.3 | 325 | 7.28E+06 | 514 | 72.5% | 2.10E+08 |
| 1-14 | citric | 2 | 150 | 1.41E+07 | 282 | 86.3% | 2.67E+08 |

Discussion of Table 2—After completion of the second cycle, the decrease in yeast cell viability (%) at 325 and 500 ppm dose continued. Also it was observed a decrease in bacteria cell counts (cells/mL) and lactic acid (mg/L) at 150, 325 and 500 ppm dose. Other parameters were not influenced by treatment.

TABLE 3
Data from Third Fermentation Cycle Experiments.

| | | | | 3th Cycle | | | | |
|---|---|---|---|---|---|---|---|---|
| | | | | Bacteria | | Lactic | Yeast | |
| | | | Dose | Count | Bacteria | Acid | Viab | Yeast UFC |
| | Acid Source | pH | (ppm) | (cells/mL) | CFU/mL | (mg/L) | (%) | (CFU/mL) |
| 1-1 | sulfuric | 3.3 | 325 | 9.55E+06 | <0.33+06 | 316.000 | 65.1% | 2.27E+08 |
| 1-2 | citric | 2 | 500 | 2.73E+06 | 6.70E+05 | 367 | 46.0% | 9.30E+07 |
| 1-3 | sulfuric | 4.5 | 150 | 1.13E+07 | 1.00E+08 | 1.680 | 87.0% | 2.53E+08 |
| 1-4 | sulfuric | 2 | 0 | 1.63E+08 | 7.00E+08 | 2.962 | 96.1% | 2.33E+08 |
| 1-5 | citric | 3.3 | 325 | 3.64E+06 | <0.33+06 | 322 | 68.4% | 2.47E+08 |
| 1-6 | citric | 4.5 | 150 | 2.04E+07 | 6.43E+07 | 1.621 | 86.4% | 2.40E+08 |
| 1-7 | sulfuric | 2 | 150 | 5.91E+06 | <0.33+06 | 312 | 83.6% | 2.47E+08 |
| 1-8 | sulfuric | 2 | 500 | 4.09E+06 | <0.33+06 | 292 | 44.9% | 1.17E+08 |
| 1-9 | citric | 3.3 | 325 | 5.91E+06 | <0.33+06 | 297 | 56.3% | 1.67E+08 |
| 1-10 | sulfuric | 2 | 0 | 2.63E+08 | 6.97E+08 | 3.634 | 95.1% | 2.70E+08 |
| 1-11 | citric | 4.5 | 500 | 1.13E+07 | <0.33+06 | 359 | 32.4% | 1.70E+08 |
| 1-12 | sulfuric | 4.5 | 500 | 4.55E+06 | <0.33+06 | 331 | 37.8% | 1.77E+08 |
| 1-13 | sulfuric | 3.3 | 325 | 5.46E+06 | <0.33+06 | 353 | 60.3% | 2.03E+08 |
| 1-14 | citric | 2 | 150 | 1.04E+06 | <0.33+06 | 261 | 76.0% | 2.07E+08 |

Discussion of Table 3—After completion of the third cycle, decrease in yeast cell viability (%) at 325 and 500 ppm dose, and the decrease in bacteria cell counts (cells/mL) and lactic acid (mg/L) at 150, 325 and 500 ppm dose was observed. Other parameters were not influenced by treatment.

TABLE 4
Data from Fourth Fermentation Cycle Experiments.

| | | | | 4th Cycle | | | |
|---|---|---|---|---|---|---|---|
| | | | | Bacteria | Lactic | Yeast | Yeast |
| | Acid | | Dose | Count | Acid | Viab | UFC |
| | Source | pH | (ppm) | (cells/mL) | (mg/L) | (%) | (CFU/mL) |
| 1-1 | sulfuric | 3.3 | 325 | 7.73E+06 | 273 | 58.8% | 1.73E+08 |
| 1-2 | citric | 2 | 500 | 1.82E+06 | 335 | 40.6% | 1.10E+08 |
| 1-3 | sulfuric | 4.5 | 150 | 9.55E+06 | 858 | 84.2% | 2.63E+08 |
| 1-4 | sulfuric | 2 | 0 | 2.07E+08 | 2.681 | 95.4% | 2.27E+08 |
| 1-5 | citric | 3.3 | 325 | 2.73E+06 | 295 | 56.9% | 1.93E+08 |
| 1-6 | citric | 4.5 | 150 | 1.72E+07 | 860 | 78.0% | 2.13E+08 |
| 1-7 | sulfuric | 2 | 150 | 4.09E+06 | 290 | 84.0% | 2.83E+08 |
| 1-8 | sulfuric | 2 | 500 | 5.46E+06 | 290 | 29.0% | 1.10E+08 |
| 1-9 | citric | 3.3 | 325 | 1.36E+06 | 251 | 47.0% | 1.63E+08 |
| 1-10 | sulfuric | 2 | 0 | 2.88E+08 | 3.364 | 93.4% | 3.00E+08 |
| 1-11 | citric | 4.5 | 500 | 7.28E+06 | 314 | 21.5% | 8.70E+07 |
| 1-12 | sulfuric | 4.5 | 500 | 4.09E+06 | 290 | 30.6% | 1.13E+08 |
| 1-13 | sulfuric | 3.3 | 325 | 5.91E+06 | 263 | 52.3% | 1.83E+08 |
| 1-14 | citric | 2 | 150 | 8.19E+06 | 240 | 83.9% | 2.60E+08 |

Discussion of Table 4—After completion of the fourth cycle, 325 and 500 ppm dose still caused decrease in yeast cell viability (%). The 150, 325 and 500 ppm dose continued to decrease bacteria cell counts (cells/mL) and lactic acid (mg/L). Other parameters were not influenced by treatment.

TABLE 5
Data from Fifth Fermentation Cycle Experiments.

| | | | | 5th Cycle | | | | |
|---|---|---|---|---|---|---|---|---|
| | | | | Bacteria | | Lactic | Yeast | |
| | Acid | | Dose | Count | Bacteria | Acid | Viab | Yeast UFC |
| | Source | pH | (ppm) | (cells/mL) | CFU/mL | (mg/L) | (%) | (CFU/mL) |
| 1-1 | sulfuric | 3.3 | 325 | 3.18E+06 | <0.33E+06 | 233 | 50.4% | 2.00E+08 |
| 1-2 | citric | 2 | 500 | 1.36E+06 | 1.00E+06 | 346 | 37.6% | 1.00E+08 |
| 1-3 | sulfuric | 4.5 | 150 | 9.10E+06 | 1.30E+07 | 526 | 89.4% | 2.50E+07 |
| 1-4 | sulfuric | 2 | 0 | 1.33E+08 | 6.25E+08 | 2.320 | 92.5% | 2.60E+08 |
| 1-5 | citric | 3.3 | 325 | 2.27E+06 | <0.33+06 | 289 | 49.0% | 1.43E+08 |
| 1-6 | citric | 4.5 | 150 | 1.04E+07 | 4.30E+05 | 399 | 82.0% | 2.10E+08 |
| 1-7 | sulfuric | 2 | 150 | 3.18E+06 | <0.33+06 | 219 | 82.6% | 2.83E+08 |
| 1-8 | sulfuric | 2 | 500 | 5.00E+06 | 6.70E+05 | 247 | 29.9% | 1.00E+08 |
| 1-9 | citric | 3.3 | 325 | 4.55E+05 | <0.33+06 | 215 | 47.0% | 1.50E+08 |
| 1-10 | sulfuric | 2 | 0 | 1.85E+08 | 7.00E+08 | 3.290 | 95.0% | 2.85E+08 |
| 1-11 | citric | 4.5 | 500 | 4.55E+06 | 1.00E+06 | 291 | 22.1% | 1.03E+08 |
| 1-12 | sulfuric | 4.5 | 500 | 3.64E+06 | <0.33+06 | 287 | 24.4% | 1.00E+08 |
| 1-13 | sulfuric | 3.3 | 325 | 5.00E+06 | <0.33+06 | 257 | 41.1% | 1.55E+08 |
| 1-14 | citric | 2 | 150 | 5.00E+06 | <0.33+06 | 199 | 70.0% | 1.97E+08 |

Discussion of Table 5—After completion of the fifth and last cycle, 325 and 500 ppm dose caused decrease in yeast cell viability (%). The 150, 325 and 500 ppm dose decreased bacteria cell counts (cells/mL) and lactic acid (mg/L). Other parameters were not influenced by treatment. As a general conclusion the best results on bacterial contamination and yeast viability was observed using 150 ppm dose with sulfuric acid, lower pH (p<0.05).

TABLE 6
Bacterial Control as a Function of Dose.

| | | Bacterial count |
|---|---|---|
| Treatment | Dose | (cells/mL) |
| Control | N/A | 4.96 × 10⁷ |
| Test dose 1 | 50 ppm | 1.58 × 10⁷ |
| Test dose 2 | 100 ppm | 2.99 × 10⁶ |
| Test dose 3 | 150 ppm | 4.55 × 10⁵ |

Discussion of Table 6—Bacterial count in broth decreased in a dose dependent manner in the presence of the test article from 50 ppm to 150 ppm.

### Example 2

The test article was evaluated for antibacterial effectiveness in a pilot plant with two 2000 L capacity fermentation vats. The pilot system included a complete fermentation temperature control system consisting of a chiller and a multi-plate heat exchanger, exclusive continuous centrifuge for biomass recycling, and tanks for storage and supply of mash for the fermentation process. The test mash included syrup, honey and water, provided by a plant, which provided the pilot system with actual processing conditions for fermentation trials. FERMEL® yeast (available from Fermentec), a commonly used yeast strain for the commercial production of ethanol, was used for the fermentation. The test article was tested at a dose of 150 ppm and compared against results obtained with Kamoran® (available from Elanco), a benchmark antibacterial product used in the control vat, at a dose of 3 ppm.

TABLE 7
Rod count in the pilot-scale trial
Contamination (rods/mL)

| Fermentation cycle | KAMORAN ®-3 ppm | Test Article-150 ppm |
|---|---|---|
| 1st Cycle | 8.50E+07 | 6.50E+07 |
| 2nd Cycle | 1.00E+05 | 1.10E+07 |
| 3rd Cycle | 1.00E+05 | 2.50E+06 |
| 4th Cycle | 1.00E+05 | 1.00E+05 |
| 5th Cycle | 1.00E+05 | 1.00E+05 |
| 6th Cycle | 2.50E+06 | 2.10E+05 |
| 7th Cycle | 1.00E+05 | 1.00E+05 |
| 8th Cycle | 1.00E+05 | 1.00E+04 |
| 9th Cycle | 4.10E+05 | 4.10E+05 |
| 10th Cycle | 8.20E+05 | 2.10E+05 |
| Mean | 8.93E+06 | 7.96E+06 |

Discussion of Table 7—Table 7 demonstrates that a single 150 ppm dose of the test article resulted in a significant reduction in bacterial cell count that after three cycles was comparable to reductions observed with Kamoran®. Moreover, the reduced bacterial counts persisted over 9 fermentation cycles, at which point Kamoran® started to lose its effectiveness.

### Example 3

The test article was evaluated for antibacterial effectiveness in an industrial setting under extreme conditions at Distillery A, which is known for having treatment resistant bacterial contamination. Seven vats having a working volume of 1 million liters each were tested in a batch fermentation process. The mash compositions included molasses and broth. The test article was tested at a dose of 150 ppm administered before the second and the seventh fermentation cycles. The vats were already at an acidic pH of less than 3 prior to the administration of the test article. The test article reduced the levels of bacterial contamination under the extreme conditions at the distillery that persisted for at least 4 fermentation cycles.

TABLE 8
Bacterial Contamination in Trial at Distillery A.

| Fermentation cycle | Contamination | Test Article |
|---|---|---|
| 1st Cycle | 5.88E+07 | |
| 2nd Cycle | 6.01E+06 | 150 ppm |
| 3rd Cycle | 3.08E+06 | |
| 4th Cycle | 5.17E+06 | |
| 5th Cycle | 1.40E+07 | |
| 6th Cycle | 5.37E+07 | |
| 7th Cycle | 1.16E+08 | 150 ppm |
| 8th Cycle | 7.73E+06 | |
| 9th Cycle | 8.42E+06 | |
| 10th Cycle | 9.60E+06 | |
| 11th Cycle | 4.21E+07 | |
| 12th Cycle | 8.14E+07 | |

The reduced bacterial contamination reduced the lactic acid balance in the vats, as illustrated in Table 9.

TABLE 9
Lactic acid balance in in Trial at Distillery A.

| | |
|---|---|
| Lactic acid balance before application | 1,486.9 kg |
| of test article | |
| Lactic acid balance in the first cycle | 463.6 kg |
| after application of test article | |

The trial at Distillery A resulted in an 80.4% reduction in bacterial contamination and an average lactic acid reduction per fermentation cycle of about 965.5 kg. The ethanol recovered as a result of this lactic acid reduction improved by 1,135.44 liters per fermentation cycle. A significant reduction of yeast cell agglomeration was also observed in the fermentation tanks.

### Example 4

The test article was evaluated for antibacterial effectiveness in an industrial setting at a second distillery—Distillery B. Six vats having a working volume of 1 million liters each were tested in a batch fermentation process. The mash compositions included molasses and broth. The test article was tested at a dose of 75 ppm administered before the second and the seventh fermentation cycles. The vats were already at an acid pH of less than 3 prior to the administration of the test article. The test article reduced the levels of bacterial contamination under the extreme conditions at the distillery that persisted for at least 4 fermentation cycles.

TABLE 10
Bacterial Contamination in Trial at Distillery B

| Fermentation | Contamination | Test Article |
|---|---|---|
| 1st Cycle | 4.68E+07 | |
| 2nd Cycle | 7.87E+06 | 75 ppm |
| 3rd Cycle | 1.89E+05 | |
| 4th Cycle | 2.19E+05 | |
| 5th Cycle | 1.54E+06 | |
| 6th Cycle | 5.05E+06 | |

The reduced bacterial contamination reduced the lactic acid balance in the vats, as illustrated in Table 11.

TABLE 11
Lactic acid balance in in Trial at Distillery B.

| | |
|---|---|
| Lactic acid balance before application | 237.6 kg |
| of test article | |
| Lactic acid balance in the first cycle | 89.9 kg |
| after application of test article | |

The trial at Distillery B resulted in a 91.9% reduction in bacterial contamination and an average lactic acid reduction per fermentation cycle of about 147.7 kg. The ethanol recovered as a result of this lactic acid reduction improved by 173.7 liters per fermentation cycle. A significant reduction of yeast cell agglomeration was also observed in the fermentation tanks.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. A method of controlling bacterial contamination in an aqueous fermentation solution employed in a sugar fermentation process, the method comprising the steps of:
(a) introducing an effective amount of a surfactant composition into the aqueous fermentation solution;
(b) introducing an acid into the aqueous fermentation solution in a sufficient quantity to provide a pH value in a range of about 4.5 or less; and
(c) mixing the aqueous fermentation solution with yeast, wherein a combination of the surfactant composition and the pH provide antibacterial effects.

2. The method of claim 1, wherein said steps are performed sequentially.

3. The method of claim 1, wherein the acid is selected from the group consisting of sulfuric acid, hydrogen sulphate, levulinic acid, caprylic acid, caproic acid, citric acid, adipic acid, tartaric acid, fumaric acid, lactic acid, phosphoric acid, hydrochloric acid, succinic acid, malic acid, sorbic acid, acetic acid, and combinations thereof.

4. The method of claim 1, wherein the pH value is in a range of about 4.5 to about 1.0.

5. The method of claim 1, wherein the pH value is in a range of about 3.5 to about 1.0.

6. The method of claim 1, wherein the surfactant composition is added in a sufficient quantity to provide a concentration of a surfactant equal to about 5 ppm or more.

7. The method of claim 1, wherein the surfactant composition is added in a sufficient quantity to provide a concentration of a surfactant equal to about 25 ppm or more.

8. The method of claim 1, wherein the surfactant composition is added in a sufficient quantity to provide a concentration of a surfactant equal to about 50 ppm or more.

9. The method of claim 1, wherein the surfactant composition is added in a sufficient quantity to provide a concentration of a surfactant equal to about 75 ppm or more.

10. The method of claim 1, wherein the surfactant composition is added in a sufficient quantity to provide a concentration of a surfactant in a range from about 75 ppm to about 350 ppm.

11. The method of one claim 1, wherein the surfactant composition comprises a non-ionic surfactant, an anionic surfactant, or a combination thereof.

12. The method of claim 1, wherein the surfactant composition comprises a surfactant selected from a group consisting of ethoxylated alcohols, ethoxylated carbohydrates, ethoxylated vegetable oils, polyethyleneglycols (PEG), polypropylene glycols (PPG), monoesters and diesters of PEG and PPG, linear or branched alkyl sulfates, ethoxylated amines, organosulfates, fatty acids, ethoxylated fatty acids, fatty amides, fatty diethanolam ides, saponin, sugar-based surfactants, and combinations thereof.

13. The method of claim 11, wherein the surfactant composition further comprises one or more acidic compounds.

14. The method of claim 1, wherein the surfactant composition comprises a surfactant selected from a group consisting of oleyl alcohol 10 EO, Tween 20, stearyl alcohol 20 EO, castor oil 80 EO, castor oil 30 EO, polyethylene glycol (PEG) 400 Dioleate, tallow amine 5 EO, Burco TME-S, coconut diethanolamide, Ethfac 161, cocoamine 2 EO, cocoamine 5 EO, Dowanol DB, Demulse DLN 532 CE, Tween 80, Demulse DLN 622 EG, Span 20, Diacid 1550, decyl alcohol 4 EO, dipropyleneglycol methyl ester, linear or branched alkyl sulfates such as sodium lauryl sulfate SLS, sodium dodecyl sulfate SDS, or sodium xylenesulfonate SXS, Tergitol NP6 and combinations thereof.

15. The method of claim 1, wherein an undesirable bacterial concentration level in the aqueous fermentation solution after a twelve hour fermentation cycle is reduced by at least a factor of 10, relative to a single twelve hour fermentation cycle of the aqueous fermentation solution at a pH equal to about 2 acidified with sulfuric acid and devoid of the surfactant composition.

16. The method of claim 1, wherein an undesirable bacterial by-product concentration level in the aqueous fermentation solution after a twelve hour fermentation cycle is reduced by 50% or more, relative to a single twelve hour fermentation cycle of the aqueous fermentation solution at a pH equal to about 1.0 acidified with sulfuric acid and devoid of the surfactant composition.

17. A method of inhibiting bacterial growth in an acidic aqueous sugar fermentation comprising mixing an aqueous fermentation solution with an effective amount of a surfactant composition, said aqueous fermentation solution comprising yeast.

18. The method of claim 17, wherein the surfactant composition comprises a surfactant selected from a group consisting of ethoxylated alcohols, ethoxylated carbohydrates, ethoxylated vegetable oils, polyethyleneglycols (PEG), polypropylene glycols (PPG), monoesters and diesters of PEG and PPG, linear or branched alkyl sulfates, ethoxylated amines, organosulfates, fatty acids, ethoxylated fatty acids, fatty amides, fatty diethanolam ides, saponin, sugar-based surfactants, and combinations thereof.

19. The method of claim 17, wherein the pH of the aqueous fermentation solution is adjusted to a desired pH with an acid selected from the group consisting of sulfuric acid, hydrogen sulphate, levulinic acid, caprylic acid, caproic acid, citric acid, adipic acid, tartaric acid, fumaric acid, lactic acid, phosphoric acid, hydrochloric acid, succinic acid, malic acid, sorbic acid, acetic acid, and combinations thereof.

20. The method of claim 17, wherein an undesirable bacterial concentration level in the aqueous fermentation solution after a twelve hour fermentation cycle is reduced by at least a factor of 10, relative to a single twelve hour fermentation cycle of the aqueous fermentation solution at a pH equal to about 2 acidified with sulfuric acid and devoid of the surfactant composition.

21. The method of claim 17, wherein an undesirable bacterial by-product concentration level in the aqueous fermentation solution after a twelve hour fermentation cycle is reduced by 50% or more, relative to a single twelve hour fermentation cycle of the aqueous fermentation solution at a pH equal to about 1.0 acidified with sulfuric acid and devoid of the surfactant composition.

22. The method of claim 17, wherein the surfactant composition comprises a surfactant selected from a group consisting of oleyl alcohol 10 EO, Tween 20, stearyl alcohol 20 EO, castor oil 80 EO, castor oil 30 EO, polyethylene glycol (PEG) 400 Dioleate, tallow amine 5 EO, Burco TME-S, coconut diethanolamide, Ethfac 161, cocoamine 2 EO, cocoamine 5 EO, Dowanol DB, Demulse DLN 532 CE, Tween 80, Demulse DLN 622 EG, Span 20, Diacid 1550, decyl alcohol 4 EO, dipropyleneglycol methyl ester, linear or branched alkyl sulfates such as sodium lauryl sulfate SLS, sodium dodecyl sulfate SDS, or sodium xylenesulfonate SXS, Tergitol NP6 and combinations thereof.

23. An aqueous fermentation solution comprising water, sugar, yeast, acid, and an antibacterial composition comprising a surfactant composition, wherein the surfactant composition is present in a sufficient quantity to provide a concentration of a surfactant equal to about 5 ppm or more; and wherein the aqueous fermentation solution has a pH value in a range of about 4.5 to about 1.0.

24. The aqueous fermentation solution of claim 21, wherein the surfactant composition comprises a surfactant selected from a group consisting of ethoxylated alcohols, ethoxylated carbohydrates, ethoxylated vegetable oils, polyethyleneglycols (PEG), polypropylene glycols (PPG), monoesters and diesters of PEG and PPG, linear or branched alkyl sulfates, ethoxylated amines, organosulfates, fatty acids, ethoxylated fatty acids, fatty amides, fatty diethanolamides, saponin, sugar-based surfactants, and combinations thereof.
